(19) United States
(12) Patent Application Publication
(10) Pub. No.: US 2026/0287567 A1
(43) Pub. Date: Sep. 24, 2026

(54) SUBSTRATE, PACKAGE, GAS SENSOR MODULE, GAS SENSOR, AND METHOD FOR MANUFACTURING GAS SENSOR

(71) Applicant: KYOCERA CORPORATION, Kyoto (JP)

(72) Inventor: Koutarou NAKAMOTO, Kyoto (JP)

(73) Assignee: KYOCERA CORPORATION, Kyoto (JP)

(21) Appl. No.: 19/469,467

(22) PCT Filed: Mar. 25, 2024
(86) PCT No.: PCT/JP2024/011720

§ 371 Date: Sep. 26, 2025

(30) Foreign Application Priority Data
Mar. 27, 2023 (JP) 2023-050547

(51) Int. Cl. G01N 33/00; G01N 27/12; H05K 1/181; H05K 3/00

(52) U.S. Cl. CPC G01N 33/0027; G01N 27/12; H05K 1/181; H05K 3/0044; H05K 2201/10151

(57) ABSTRACT

To simplify and miniaturize the structure of a gas sensor. The substrate includes a base including a plurality of insulation layers made of a ceramic material and including a first surface and a second surface located on a side opposite to the first surface, a gas sensitive body region located on the first surface and in which a gas sensitive body is disposed, an electrode wiring located in the gas sensitive body region and connected to the gas sensitive body, a heater located between the insulation layers and located overlapping the gas sensitive body region in a perspective plan view, and an external electrode electrically connected to the electrode wiring or the heater.

### TECHNICAL FIELD

The present disclosure relates to a substrate, a package, a gas sensor, and a method for manufacturing a gas sensor.

### BACKGROUND OF INVENTION

A known small gas sensor includes a CAN type gas sensor as disclosed in Patent Document 1 or a gas sensor mounted with a MEMS element as disclosed in Patent Document 2.

### CITATION LIST

### Patent Literature

Patent Document 1: JP S63-275941 A
Patent Document 2: JP 2018-100900 A

### SUMMARY

A substrate according to an aspect of the present disclosure includes a base including a plurality of insulation layers made of a ceramic material and including a first surface and a second surface located on a side opposite to the first surface, a gas sensitive body region located on the first surface and in which a gas sensitive body is disposed, an electrode wiring located in the gas sensitive body region and connected to the gas sensitive body, a heater located between the insulation layers and located overlapping the gas sensitive body region in a perspective plan view, and an external electrode electrically connected to the electrode wiring or the heater.

In an aspect of the present disclosure, a package includes the substrate and a lid.

In an aspect of the present disclosure, a gas sensor includes the package and a gas sensitive body.

In an aspect of the present disclosure, a gas sensor module includes the gas sensor and a mounting substrate, and the gas sensor is surface-mounted on the mounting substrate by soldering.

In an aspect of the present disclosure, a method for manufacturing a gas sensor includes preparing a mother board including a plurality of substrate regions corresponding to the substrate, forming a gas sensitive body in the gas sensitive body region, and singulating the mother board.

### BRIEF DESCRIPTION OF THE DRAWINGS

FIG. is a cross-sectional view of a gas sensor according to a first embodiment of the present disclosure.

FIG. 2 is a top view of a substrate according to the first embodiment of the present disclosure.

FIG. 3 is a perspective plan view illustrating a configuration of a heater surface of the substrate according to the first embodiment of the present disclosure.

FIG. 4 is a bottom view of a substrate according to the first embodiment of the present disclosure.

FIG. 5 is a top view of a lid according to the first embodiment of the present disclosure.

FIG. 6 is a cross-sectional view of a gas sensor module including the gas sensor according to the first embodiment of the present disclosure.

FIG. 7 is a cross-sectional view of a substrate according to a second embodiment of the present disclosure.

FIG. 8 is a top view of the substrate according to the second embodiment of the present disclosure.

FIG. 9 is a perspective plan view illustrating a configuration of a heater surface of the substrate according to the second embodiment of the present disclosure.

FIG. 10 is a horizontal cross-sectional view of the substrate according to the second embodiment of the present disclosure.

FIG. 11 is a perspective plan view illustrating a configuration of a heater surface illustrating another embodiment of a cavity.

FIG. 12 is a perspective plan view illustrating a configuration of a heater surface illustrating another embodiment of a cavity.

FIG. 13 a perspective plan view illustrating a configuration of a heater surface illustrating another embodiment of a cavity.

FIG. 14 is a cross-sectional view illustrating another embodiment of a cavity.

FIG. 15 is a cross-sectional view of a substrate according to a third embodiment of the present disclosure.

FIG. 16 is a top view of the substrate according to the third embodiment of the present disclosure.

FIG. 17 is a cross-sectional view of a substrate according to a fourth embodiment of the present disclosure.

FIG. 18 is a top view of the substrate according to the fourth embodiment of the present disclosure.

FIG. 19 is a cross-sectional view of a gas sensor according to a fifth embodiment of the present disclosure.

FIG. 20 is a top view of a substrate according to the fifth embodiment of the present disclosure.

FIG. 21 is a top view of a lid according to the fifth embodiment of the present disclosure.

FIG. 22 is a cross-sectional view of a gas sensor according to a sixth embodiment of the present disclosure.

FIG. 23 is a cross-sectional view of a gas sensor according to a seventh embodiment of the present disclosure.

FIG. 24 is a cross-sectional view of a gas sensor according to an eighth embodiment of the present disclosure.

FIG. 25 is a top view of a substrate according to the eighth embodiment of the present disclosure.

FIG. 26 is a perspective view of the gas sensor according to the eighth embodiment of the present disclosure.

FIG. 27 is a cross-sectional view of a gas sensor according to a ninth embodiment of the present disclosure.

FIG. 28 is a top view of a substrate according to the ninth embodiment of the present disclosure.

FIG. 29 is a cross-sectional view of the gas sensor according to the ninth embodiment of the present disclosure.

FIG. 30 is a cross-sectional view of a gas sensor according to a tenth embodiment of the present disclosure.

FIG. 31 is a cross-sectional view of another gas sensor according to the tenth embodiment of the present disclosure.

FIG. 32 is a cross-sectional view of another gas sensor according to the tenth embodiment of the present disclosure.

FIG. 33 is a cross-sectional view of another gas sensor according to the tenth embodiment of the present disclosure.

FIG. 34 is a cross-sectional view of another gas sensor according to the tenth embodiment of the present disclosure.

FIG. 35 is a top view of another gas sensor according to the tenth embodiment of the present disclosure.

FIG. 36 is a cross-sectional view of a gas sensor according to an eleventh embodiment of the present disclosure.

FIG. 37 is a top view of a substrate according to the eleventh embodiment of the present disclosure.

FIG. 38 is a cross-sectional view of a gas sensor module and an electronic apparatus including the gas sensor according to the eleventh embodiment of the present disclosure.

FIG. 39 is a schematic plan view illustrating a part of a mother board according to a twelfth embodiment of the present disclosure.

FIG. 40 is a cross-sectional view of a gas sensor according to a thirteenth embodiment of the present disclosure.

FIG. 41 is a top view of the gas sensor according to the eleventh embodiment of the present disclosure.

### DESCRIPTION OF EMBODIMENTS

The CAN type gas sensor has a complicated manufacturing step and is difficult to miniaturize. The gas sensor mounted with a MEMS element is mounted with an expensive and precise MEMS chip, and when manufacturing the gas sensor, the MEMS chip and a wiring substrate need to be connected to each other by wire bonding or the like.

An aspect of the present disclosure realizes simplification and miniaturization of a structure of a gas sensor.

According to an aspect of the present disclosure, the structure of the gas sensor can be simplified and miniaturized.

### First Embodiment

An embodiment of the present disclosure is described below in detail.

The distinction between top and bottom in the following description is for convenience only and does not limit the actual top and bottom of a substrate, a package and a gas sensor when they are used. In this description, an upper surface is defined as a surface on which a gas sensitive body 8 is provided in a substrate 10. In the drawings, the positive Z axis direction is the upward direction. The X axis direction is a direction parallel to any one side of the substrate 10, and the Y axis is an axis that perpendicularly intersects the X axis and Z axis. The thickness direction of the substrate 10 is the Z axis direction. The dimensions of the components in the drawings may not faithfully represent the actual dimensions of the components, the dimension ratios of the members, or the like. In the present disclosure, the rectangular shape is not limited to a strict rectangular shape, and includes a shape that can be visually recognized as a rectangular shape as a whole even if corner portions are curved, for example.

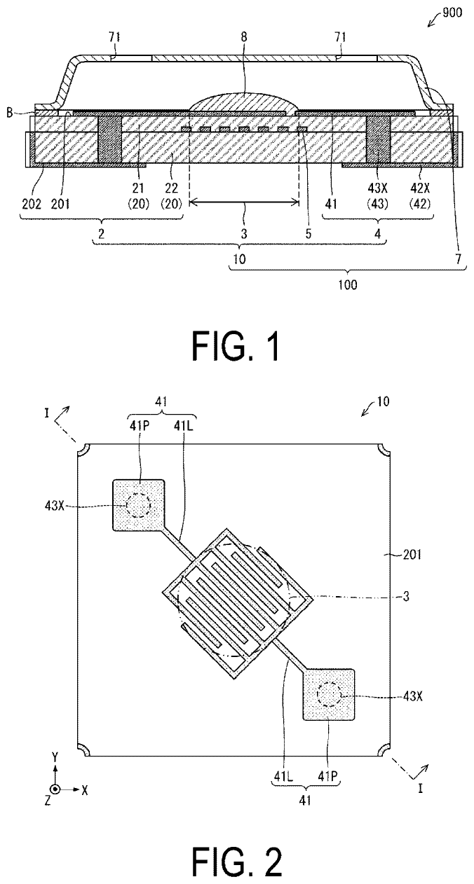

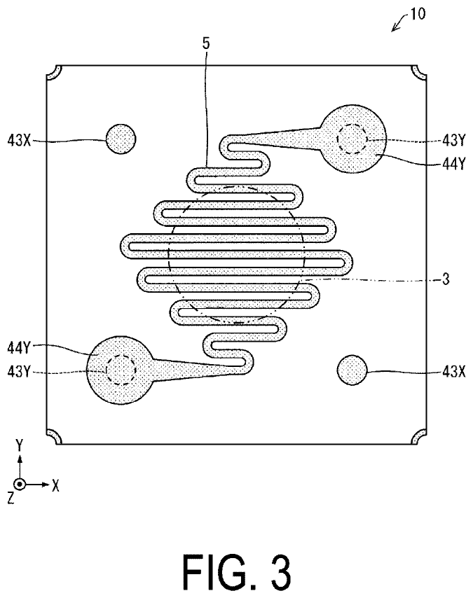

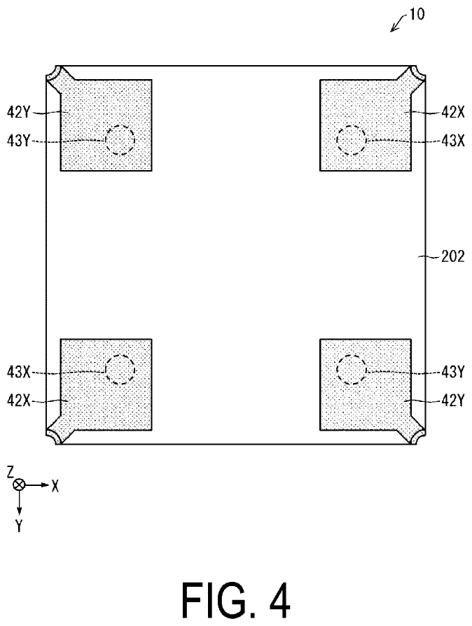

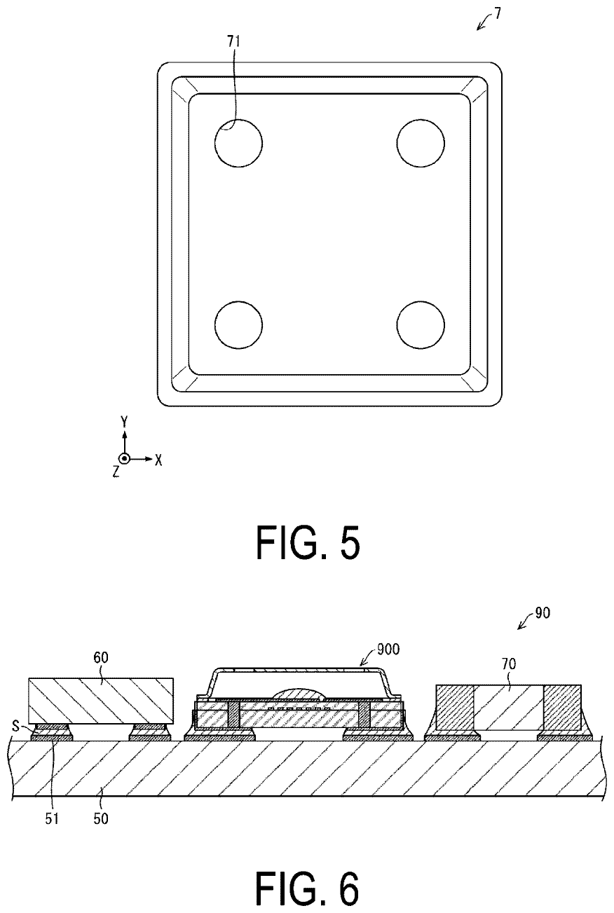

FIG. 1 is a cross-sectional view of a gas sensor 900 according to the first embodiment, and illustrates a cross-sectional view taken along the arrow line I-I in a state where a bonding material B and a lid 7 are mounted to the substrate 10 illustrated in FIG. 2. FIG. 2 is a top view of the substrate 10. FIG. 3 is a perspective plan view illustrating a configuration of a heater surface of the substrate 10. The heater surface is a surface on which a heater 5 described later is located. In FIG. 3, a virtual region of a gas sensitive body region 3 is illustrated for the sake of easy description. FIG. 4 is a bottom view of the substrate 10. FIG. 5 is a top view of the lid 7. In the present description, a plan view when viewed from the Z axis positive direction may be referred to as a top view, and a plan view when viewed from the Z axis negative direction may be referred to as a bottom view.

The present disclosure relates to a substrate-type gas sensor. The gas sensor 900 according to the first embodiment includes a package 100 and the gas sensitive body 8. The package 100 includes the substrate 10 and the lid 7. The substrate 10 includes a base 2, the gas sensitive body region 3, a wiring 4, and the heater 5.

### Substrate

The substrate 10 is a support substrate of the gas sensitive body 8 and also has a function as a wiring substrate. Members constituting the substrate 10 will be described in detail below.

The base body 2 is a body portion of the substrate 10 and includes a plurality of insulation layers 20 layered in the thickness direction of the substrate 10. The insulation layer 20 is made of a ceramic material. The substrate 10 includes a first surface 201 located in the layered direction and a second surface 202 located opposite to the first surface 201. Among the insulation layers 20 included in the base 2, an insulation layer including the first surface 201 is referred to as a first insulation layer 21. Among the insulation layers 20 included in the base 2, an insulation layer layered on the first insulation layer 21 is referred to as a second insulation layer 22. The second insulation layer 22 is an insulation layer layered on the first insulation layer 21 on the side opposite to the first surface 201. The first insulation layer 21 may be an insulation layer group that is located above the heater 5 and includes the plurality of insulation layers 20. The second insulation layer 22 may be an insulation layer group that is located on the opposite side of the first insulation layer 21 with the heater 5 interposed therebetween and includes the plurality of insulation layers 20.

As illustrated in FIG. 2, the shape of the base 2 in a plan view may be rectangular. The base 2 is made of a ceramic such as an aluminum oxide sintered body (alumina ceramic), an aluminum nitride sintered body, a mullite sintered body, or a glass ceramic sintered body. The shape of the base 2 in a plan view according to the first embodiment is substantially square, but may be rectangular. As illustrated in FIG. 2, the base 2 may include a cutout at a corner portion. The shape of the base 2 in a plan view is not limited to a rectangle, and may be changed as appropriate depending on the application or the like.

The gas sensitive body region 3 is a region that is located on the first surface 201 and in which the gas sensitive body 8 is disposed. The gas sensitive body region 3 may be a region overlapping the gas sensitive body 8 disposed in the gas sensitive body region 3 on the first surface 201. Alternatively, the gas sensitive body region 3 may be a region surrounded by virtual lines connecting alignment marks that are located on the first surface 201 and used when disposing the gas sensitive body 8.

The wiring 4 includes an electrode wiring 41, an external electrode 42, and a through conductor 43. The wiring 4 may include an interlayer conductor 44. The through conductor 43 penetrates at least one insulation layer 20 in the vertical direction and connects the wirings located above and below the through conductor 43. The wiring connected via the through conductor 43 may be the electrode wiring 41, the external electrode 42, or the interlayer conductor 44. The interlayer conductor 44 is a conductor located between the insulation layers 20.

The electrode wiring 41 is a wiring at least a part of which is located in the gas sensitive body region 3 and which is connected to the gas sensitive body 8. As illustrated in FIG. 2, the electrode wiring 41 may include a first portion 41P and a second portion 41L. The first portion 41P and the second portion 41L may be continuously integrated. The first portion 41P and the second portion 41L may be metallized patterns formed by printing and sintered simultaneously with the base 2 made of ceramics. Alternatively, the first portion 41P and the second portion 41L may be metallized patterns formed by printing or vaporizing after the base 2 made of ceramics is sintered.

The first portion 41P may be a conductor having a plate shape. The first portion 41P is a portion that functions as a connection pad for connecting to the through conductor 43X, and has an area larger than the radius of the through conductor 43X in a plan view. As illustrated in FIG. 2, the first portion 41P is connected to the through conductor 43X that is located overlapping the first portion 41P and penetrate the base 2 in a perspective plan view. The through conductor 43X is connected to a first external electrode 42X. As illustrated in FIG. 4, the through conductor 43X is located overlapping the first external electrode 42X in a perspective plan view. The first portion 41P may have a rectangular shape with a side length larger than the diameter of the through conductor 43 in a cross section perpendicular to the Z axis. The shape of the first portion 41P is not limited to a rectangular shape, and may be any shape. For example, the first portion 41P may have a circular shape having a diameter larger than the diameter of the cross section of the through conductor 43. In the substrate 10 illustrated in FIG. 2, the two first portions 41P are located along a diagonal line of the substrate 10. The arrangement of the first portions 41P is not limited to this arrangement, and for example, the two first portions 41P may be disposed along any one side of the substrate 10 having a rectangular shape.

The second portion 41L is at least partially located in the gas sensitive body region 3 and includes an extension portion extending from the gas sensitive body region 3 to the first portion 41P. The second portion 41L may have a comb-tooth shape in a portion located in the gas sensitive body region 3. The shape of the portion located in the gas sensitive body region 3 is not limited to the comb-tooth shape. Alternatively, the second portion 41L may have a single band shape extending from the gas sensitive body region 3 to the first portion 41P.

In the substrate 10, the external electrode 42 is located on the second surface 202 of the base 2 and is electrically connected to the electrode wiring 41 or the heater 5. The external electrode 42 is located on the second surface 202, thus enabling surface mounting. The external electrode 42 may include, for example, two first external electrodes 42X electrically connected to the two respective electrode wirings 41 and two second external electrodes 42Y electrically connected to the two respective terminals of the heater 5, as illustrated in FIG. 4. The first external electrodes 42X are electrically connected to the electrode wirings 41 via the through conductors 43X. The second external electrodes 42Y are electrically connected to the heater 5 via the through conductors 43Y. In the substrate 10, the two first external electrodes 42X are located along a diagonal line of the substrate 10. In other words, the two first external electrodes 42X are located at opposite corner portions of the base 2. The arrangement of the first external electrodes 42X is not limited to this arrangement. For example, the two first external electrodes 42X may be disposed along any one side of the substrate 10 having a rectangular shape.

When the gas sensor 900 is mounted on a mounting substrate such as a printed circuit board (PCB), the external electrode 42 is connected to a circuit substrate (external electric circuit) of the device via a solder material. In the substrate 10, the external electrode 42 may be provided from the second surface 202 to the side surface of the base 2. As illustrated in FIGS. 1 and 2, when the substrate 10 includes a cutout portion at a corner portion, the external electrode 42 located on the side surface may be located in the cutout portion.

The electrode wiring 41, the external electrode 42, the through conductor 43, and the interlayer conductor 44 mainly contain, for example, metal such as tungsten, molybdenum, manganese, copper, silver, palladium, gold, platinum, nickel, and cobalt, or an alloy containing any of these metals as a conductor material. The electrode wiring 41 and the external electrode 42 are formed on the surfaces of the body 2 as metal layers such as metallization layers or plating layers of a conductor material. The metal layer may be a single layer, or a plurality of layers. The interlayer conductor 44 is formed inside the base 2 by metallization of a conductor material.

For example, when the electrode wiring 41 and the external electrode 42 are metallization layer of tungsten, they can be formed by applying a metal paste made by mixing tungsten powder with an organic solvent and organic binder at predetermined positions of the insulation layer 20 by a screen printing method or the like. The through conductor 43 may be formed by providing a through hole at a predetermined position in the ceramic green sheet prior to printing of the metal paste above and filling this through hole with a metal paste that is the same as or similar to the metal paste above.

An exposed surface of the metallization layer after firing may be further coated with a plating layer of nickel, gold, or the like using an electrolytic plating method or an electroless plating method.

The heater 5 is located between the first insulation layer 21 and the second insulation layer 22. Generally, the gas sensitive body 8 detects gas in a state of being heated to a temperature of approximately 200° C. to 500° C., but this varies depending on the types of gas detected. The heater 5 is for heating the gas sensitive body 8. The heater 5 may be a heater circuit pattern as illustrated in FIG. 3. The heater 5 is connected to heater terminals 44Y located in the same layer as the heater 5. As illustrated in FIG. 3, the heater 5 and the heater terminals 44Y may be integrally formed. The heater terminals 44Y are an example of the interlayer conductors 44. The heater terminals 44Y are connected to through conductors 43Y that are located overlapping the heater terminals 44Y in a perspective plan view and penetrate the second insulation layer 22. The heater terminals 44Y are portions that function as connection pads for connecting to the through conductors 43Y, and have areas larger than the diameters of the through conductors 43Y in a plan view. The planar shape of the heater terminals 44Y may be circular as illustrated in FIG. 3, or may be another shape such as a rectangle. The through conductors 43Y are located overlapping the second external electrodes 42Y in a perspective plan view and are connected to the second external electrodes 42Y.

In order to improve the heating efficiency of the gas sensitive body 8 by the heater 5, the first insulation layer 21 may be thinner than the second insulation layer 22. In other words, the distance between the heater 5 and the first surface 201 may be smaller than the distance between the heater 5 and the second surface 202. Since the first insulation layer 21 is thinner than the second insulation layer 22, the heat from the heater 5 is more easily transferred to the first surface side on which the gas sensitive body 8 is located than to the second surface side connected to the outside.

The distance between the heater 5 and the first surface 201 may be 10 μm or more and 100 μm or less. In other words, the thickness of the first insulation layer 21 may be 10 μm or more and 100 μm or less. Since the thickness of the first insulation layer 21 is 100 μm or less, the heat of the heater 5 is easily transferred to the gas sensitive body 8. Since the thickness of the first insulation layer 21 is 10 μm or more, the possibility that the heater is exposed to the first surface 201 can be reduced. Since the thickness of the first insulation layer 21 is 10 μm or more, the electrical insulation between the heater 5 and the electrode wiring 41 can be secured.

On the other hand, the external electrode 42 located on the second surface 202 is bonded to a circuit electrode of a mounting substrate via solder or an electrically conductive bonding material made of resin. Since the heat from the heater 5 is more easily transferred to the first surface side on which the gas sensitive body 8 is located than to the second surface side, the influence of the heat exerted on the electrically conductive bonding material can be reduced by locating the external electrode 42 on the second surface 202. Therefore, for example, the distance between the heater 5 and the second surface 202 may be 100 μm or more. In other words, the thickness of the second insulation layer 22 may be 100 μm or more. Since the thickness of the second insulation layer 22 is 100 μm or more, the influence of heat exerted on the electrically conductive bonding material by the heater 5 can be reduced.

### Gas Sensitive Body

The gas sensitive body 8 includes a metal oxide semiconductor material corresponding to the type of the gas sensor 900. Examples of the metal oxide semiconductor material include a material containing one or more types selected from tin oxide (SnO₂ or the like), indium oxide (In₂O₃ or the like), zinc oxide (ZnO or the like), tungsten oxide (WO₃ or the like), iron oxide (Fe₂O₃ or the like), and the like. By appropriately adding impurities to the metal oxide semiconductor material of the gas sensitive body 8, the gas to be detected by the gas sensor can be appropriately selected. The gas sensitive body 8 detects the gas to be detected based on a change in resistance value due to contact with the gas to be detected.

### Lid

In the gas sensor 900, the gas sensitive body 8 is protected by the lid 7. In the example illustrated in FIG. 1, the gas sensor 900 includes the cap-shaped lid 7 that is located on the first surface 201 of the substrate 10 and collectively covers the electrode wiring 41 and the gas sensitive body 8. As illustrated in FIGS. 1 and 5, the lid 7 may include a through hole 71 for ventilation. The lid 7 may include a plurality of through holes 71. With the plurality of through holes, breathability can be improved. The through holes 71 may be provided at positions not overlapping the gas sensitive body 8 in a perspective plan view. Not overlapping the gas sensitive body 8 in a perspective plan view can reduce the possibility that dust or the like that has entered from the through holes 71 adheres to the gas sensitive body 8.

The lid 7 may be made of, for example, ceramic or metal. FIG. 1 illustrates an example in which the lid 7 is made of metal. Since the lid 7 is made of metal, the cap-shaped lid can be easily manufactured by bending. The lid 7 can be formed to be thinner than when the lid 7 is made of a ceramic material, and the package 100 and the gas sensor 900 can be further miniaturized.

The lid 7 made of metal and the substrate 10 may be bonded to each other by, for example, a resin. That is, the bonding material B may be a resin. Alternatively, the lid 7 and the substrate 10 may be bonded to each other by solder bonding or brazing bonding. These bonds are made by bulk heating using a batch furnace, tunnel furnace, or the like. In the case of solder bonding or brazing bonding, a frame-shaped metal film may be located on the first surface 201, and the lid 7 may be bonded onto the frame-shaped metal film. The frame-shaped metal film may be formed on the first surface 201 by metallization.

Direct seam welding, laser welding, or electron beam welding may be used to bond the metal lid 7 and the frame-shaped metal film. In these welding methods, since bonding is performed by local heating to a bonding portion, sealing can be performed at a lower temperature than the bulk heating. When the lid 7 is made of metal, an iron-nickel (Fe—Ni) alloy or an iron-nickel-cobalt (Fe—Ni—Co) alloy may be used as the material of the lid 7. These alloys are suitable for the lid 7 of the substrate 10 because of a small difference in thermal expansion from ceramic.

As described above, the substrate 10 includes the base 2 including the plurality of insulation layers 20 made of a ceramic material and including the first surface 201 and the second surface 202 located on the side opposite to the first surface 201. The substrate 10 includes the gas sensitive body region 3 which is located on the first surface 201 and in which the gas sensitive body 8 is disposed. The substrate 10 includes the electrode wiring 4 which is located in the gas sensitive body region 3 and connected to the gas sensitive body 8. The substrate 10 includes the heater 5 located between the first insulation layer 21 and the second insulation layer 22 and located overlapping the gas sensitive body region 3 in a perspective plan view. The substrate 10 includes the external electrode 42 electrically connected to the electrode wiring 41 or the heater 5.

Since the substrate 10 has a configuration in which the gas sensitive body can be directly disposed on the wiring substrate, the structure of the gas sensor manufactured using the substrate 10 can be simplified and miniaturized. The simplification of the structure can simplify the manufacturing step, which contributes to the reduction of the manufacturing cost. Since the substrate 10 does not additionally require a micro electro mechanical systems (MEMS) including a built-in heater or an alumina substrate on which a heater circuit is printed, and the heater is built in the wiring substrate itself, the wiring substrate can serve as a gas sensor device by applying the gas sensitive body.

The package 100 includes the substrate 10 and the lid 7, and thus can protect the gas sensitive body 8. The package 100 can simplify the structure of the gas sensor manufactured using the package 100 and miniaturize the gas sensor.

The gas sensor 900 includes the package 100 and the gas sensitive body 8, and thus can be miniaturized compared to a conventional gas sensor. The gas sensor 900 has a configuration that can be modularized by surface mounting. FIG. 6 is a cross-sectional view of a gas sensor module 90 including the gas sensor 900. The gas sensor module 90 includes a mounting substrate 50 and the gas sensor 900. In the example illustrated in FIG. 6, the gas sensor 900, a semiconductor IC 60, and a capacitor 70 are mounted on the mounting substrate 50. The semiconductor IC 60 may be, for example, an application specific integrated circuit (ASIC). The gas sensor 900, the semiconductor IC 60, and the capacitor 70 are surface-mounted on the mounting substrate 50 by solder S, respectively. As illustrated in FIG. 6, the gas sensor 900 can be directly soldered onto the mounting substrate together with electronic components such as the semiconductor IC and the capacitor to form the gas sensor module 90.

### Second Embodiment

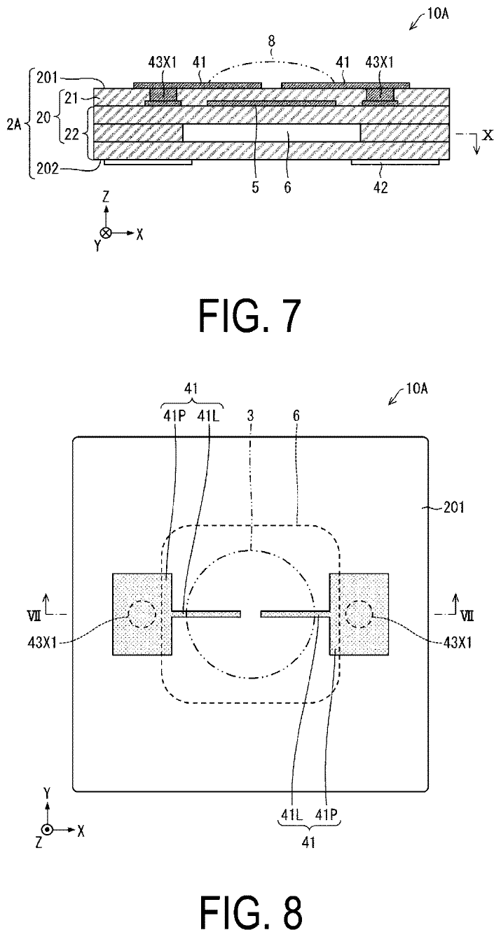

Another embodiment of the present disclosure will be described below. For convenience of description, members having the same functions as those of the members described in the above-described embodiment are denoted by the same reference signs, and description thereof is not repeated. In the second embodiment, first, a substrate 10A according to another embodiment will be described with reference to FIGS. 7 to 10.

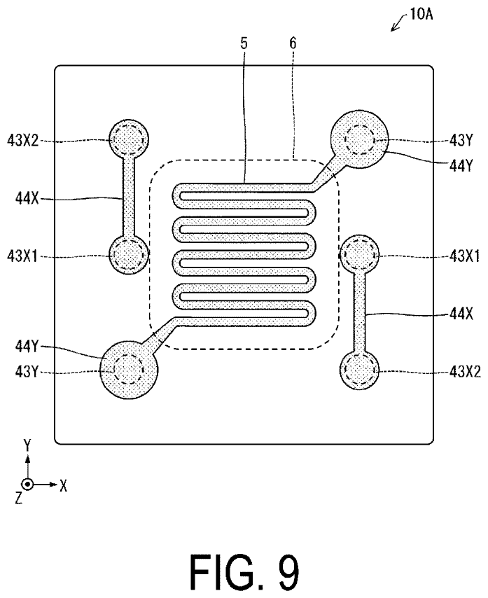

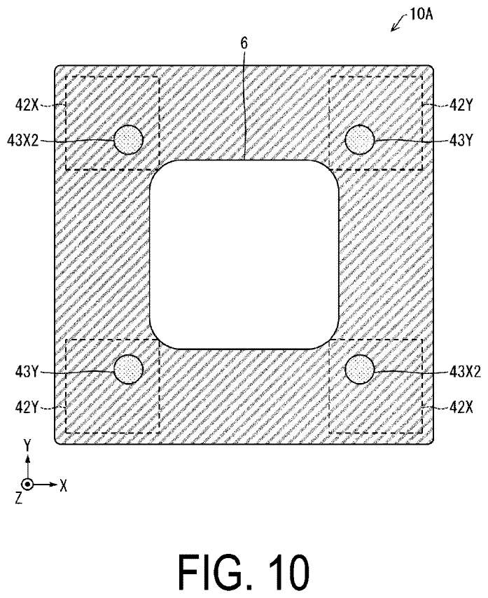

FIG. 7 is a cross-sectional view of the substrate 10A according to the second embodiment, and is a cross-sectional view taken along the arrow line VII-VII in FIG. 8. FIG. 8 is a top view of the substrate 10A. FIG. 9 is a perspective plan view illustrating a configuration of a heater surface of the substrate 10A. FIG. 10 is a cross-sectional view of the substrate 10A and is a cross-sectional view taken along the arrow line X-X in FIG. 7. In FIGS. 8, 9, and 10, the shapes of the members and the cavities in a perspective plan view are indicated by dotted lines as appropriate for easy understanding of the positional relationship.

The substrate 10A is different from the substrate 10 of the first embodiment in that a cavity 6 is provided in the substrate 10A. The substrate 10A includes a base 2A. The substrate 2A includes the cavity 6 between the heater 5 and the second surface 202. To be more specific, the base 2A has a hollow structure including the cavity 6 inside the second insulation layer 22. The cavity 6 is located overlapping the heater 5 in a top perspective view. In a perspective plan view, the outer edge of the cavity 6 may be larger than the heater 5. Since the cavity 6 is located between the heater 5 and the external electrode 42, heat conduction to the bonding material or solder and the external terminal is reduced. As illustrated in FIG. 7, the second insulation layer 22 is an insulation layer group including a plurality of insulation layers 20, and an insulation layer 20 located in the middle of the plurality of insulation layers 20 may include a through hole. The cavity 6 can be formed by the through hole and the insulation layers 20 located above and below the through hole. The shape of the cavity 6 may be rectangular in a plan view, as illustrated in FIGS. 7 to 10. The shape of the cavity 6 is not limited to the rectangular shape.

As illustrated in FIG. 8, the second portion 41L of the electrode wiring 41 may have a single band shape extending from the gas sensitive body region 3 to the first portion 41P. The thickness of the second portion 41L can be appropriately designed in consideration of resistance, a design allowable range, and the like. As illustrated in FIG. 8, the two first portions 41P located on the first surface 201 may be disposed to be substantially line-symmetric with respect to a center line connecting the centers of the facing sides in a plan view.

As illustrated in FIGS. 7 and 8, the first portions 41P of the electrode wiring are connected to through conductors 43X1 that are located overlapping the first portions 41P and penetrate the first insulation layer 21 in a perspective plan view. As illustrated in FIG. 9, the through conductors 43X1 are connected to through conductors 43X2 via interlayer conductors 44X. The through conductors 43X1 and the through conductors 43X2 are located inside the interlayer conductors 44X in a perspective plan view. In order to improve the connection reliability, the area of the interlayer conductor 44X at the bonding surface between the interlayer conductor 44X and the through conductor 43X1 or the through conductor 43X2 may be larger than the area of the through conductor 43X1 or the through conductor 43X2. As illustrated in FIG. 10, the through conductors 43X2 are conductors that are located overlapping the first external electrodes 42X in a perspective plan view and penetrate the second insulation layer 22. The through conductors 43X2 are connected to the first external electrodes 42X.

By including the cavity 6 between the heater 5 and the second surface 202 as in the substrate 10A, heat conduction from the heater to the second surface 202 can be reduced, and the possibility of re-melting of the solder bonded to the mounting substrate in the secondary mounting can be reduced. Since the heat from the heater 5 is more easily conducted to the gas sensitive body 8 side than to the cavity 6 having a low heat conductivity, the heat from the heater 5 can be efficiently conducted to the gas sensitive body 8.

Since the substrate 10A has a hollow structure, even when the cavity 6 is provided below the heater 5, the area of the second surface 202 can be secured. This increases the degree of freedom in arrangement of the external electrode 42 located on the second surface 202. Since the area of the external electrode 42 can be increased, the mounting reliability is improved.

### Other First Embodiment of Cavity 6

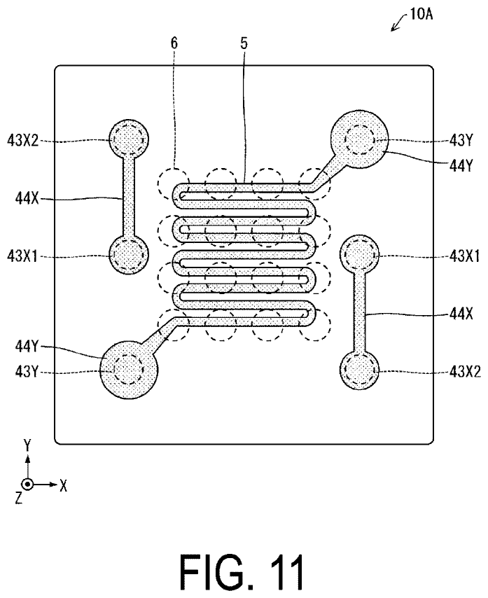

FIGS. 11 to 14 are diagrams illustrating other embodiments of the cavity 6. FIGS. 11 to 13 are perspective plan views illustrating configurations of a heater surface. FIG. 14 is a cross-sectional view of the same cross section as FIG. 7. In FIGS. 11 to 13, the shapes of the members and the cavities in a perspective plan view are indicated by dotted lines as appropriate for easy understanding of the positional relationship.

As illustrated in FIG. 11, the substrate 10A may include a plurality of cavities 6 at positions overlapping the heater 5 in a perspective plan view. In the example illustrated in FIG. 11, the second insulation layer 22 is an insulation layer group including a plurality of insulation layers 20, and the insulation layer 20 located in the middle of the plurality of insulation layers 20 may include a plurality of through holes. The plurality of through holes may be located in different insulation layers 20. The shape of the cavity 6 may be circular in a plan view as illustrated in FIG. 11. The entire plurality of through holes may be larger than the heater 5. In other words, in a perspective plan view, the plurality of through holes may be arranged in a range wider than the heater 5.

Since the substrate 10A includes the plurality of cavities 6, a part of the insulation layer 20 is located between the cavities 6, and a decrease in the strength and rigidity of the substrate due to the cavities 6 can be reduced. The possibility of deformation occurring during the manufacture of the substrate 10A including the insulation layer 20 made of a ceramic material can be reduced.

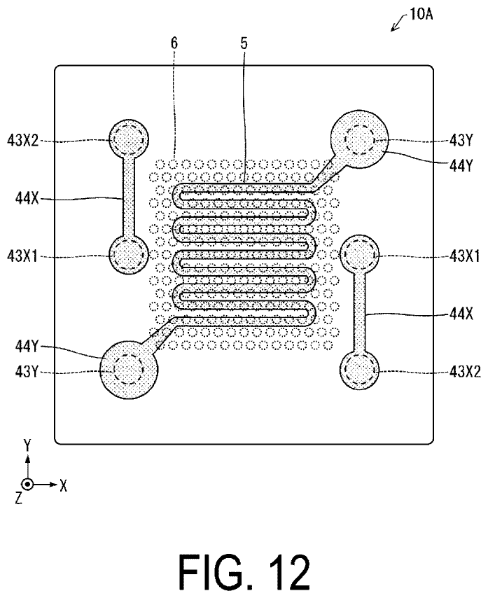

As illustrated in FIG. 12, the substrate 10A may include a large number of fine cavities 6 at positions overlapping the heater 5 in a perspective plan view. In the example illustrated in FIG. 12, the second insulation layer 22 is an insulation layer group including a plurality of insulation layers 20, and the insulation layer 20 located in the middle of the plurality of insulation layers 20 may include a plurality of through holes. The plurality of through holes may be located in different insulation layers 20.

Since the substrate 10A includes the large number of fine cavities 6, the decrease in the strength and rigidity of the substrate due to the cavities 6 can be further reduced. The possibility of deformation occurring during the manufacture of the substrate 10A including the insulation layer 20 made of a ceramic material can be further reduced.

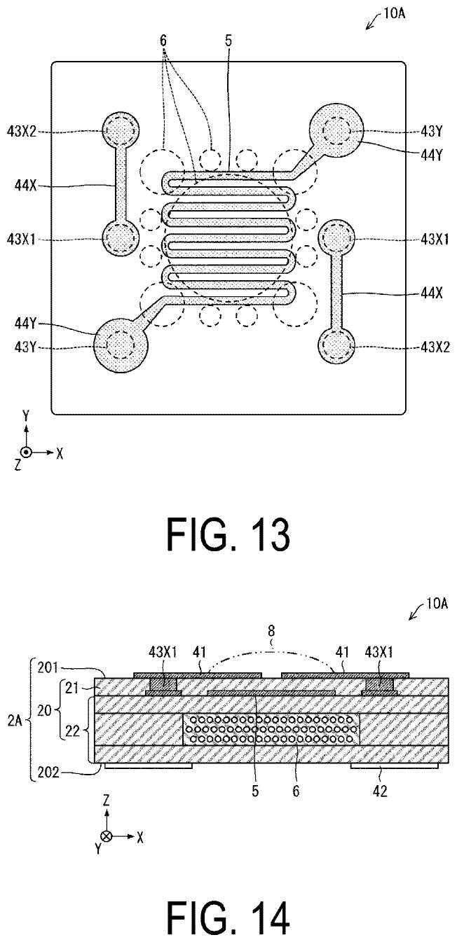

As illustrated in FIG. 13, the substrate 10A may include a plurality of cavities 6 having different sizes in a plan view at positions overlapping the heater 5 in a perspective plan view. The plurality of cavities 6 include a large-diameter cavity 6 which has a size similar to that of the gas sensitive body region 3 and is located at a position overlapping the gas sensitive body region 3, and a small-diameter cavity 6 around the large-diameter cavity 6. The heat is efficiently conducted to the gas sensitive body region 3 by the large-diameter cavity 6 located on the opposite side of the gas sensitive body region 3 with the heater 5 interposed therebetween, and the plurality of cavities 6 are dispersedly disposed, whereby the reduction in the strength and rigidity of the substrate due to the cavities 6 can be reduced. Among the small-diameter cavities 6, the cavity 6 located between the heater 5 and the external electrode 42 is larger than the other cavities 6, and thus the heat conduction to the external electrode 42 can be more effectively reduced.

Even when the plurality of cavities 6 have different sizes, the decrease in the strength and rigidity of the substrate due to the cavities 6 can be reduced. The possibility of deformation occurring during the manufacture of the substrate 10A including the insulation layer 20 made of a ceramic material can be reduced.

Alternatively, as illustrated in FIG. 14, the substrate 10A may include a large number of micro-voids disposed three dimensionally inside the base 2A. The micro-voids are an example of the cavities 6. The above-described cavity 6 is a through hole penetrating the insulation layer 20, whereas each of the large number of micro-voids does not penetrate the insulation layer 20. In other words, the height of the micro-voids is significantly small compared to the thickness of the insulation layer 20. A plurality of micro-voids are present in the thickness direction of one insulation layer 20. The region where the large number of micro-voids are disposed may be a position overlapping the heater 5 in a perspective plan view. The large number of micro-voids disposed three dimensionally can be formed by, for example, filling a through hole for forming the cavity 6 illustrated in FIG. 7 with a ceramic green sheet, a ceramic paste, or the like containing a large amount of a binder or the like, and firing them.

Since the substrate 10A includes a large number of micro-voids, a decrease in the strength and rigidity of the substrate due to the cavity 6 can be reduced. The possibility of deformation occurring during the manufacture of the substrate 10A including the insulation layer 20 made of a ceramic material can be reduced.

Alternatively, a layer made of a ceramic material having a lower heat conductivity than the other layers may be used between the heater 5 and the external electrode 42. This makes it difficult for heat to be transferred to the solder or the bonding material bonded to the external electrode 42 and the external terminal, and thus the possibility of re-melting of the solder can be reduced.

### Third Embodiment

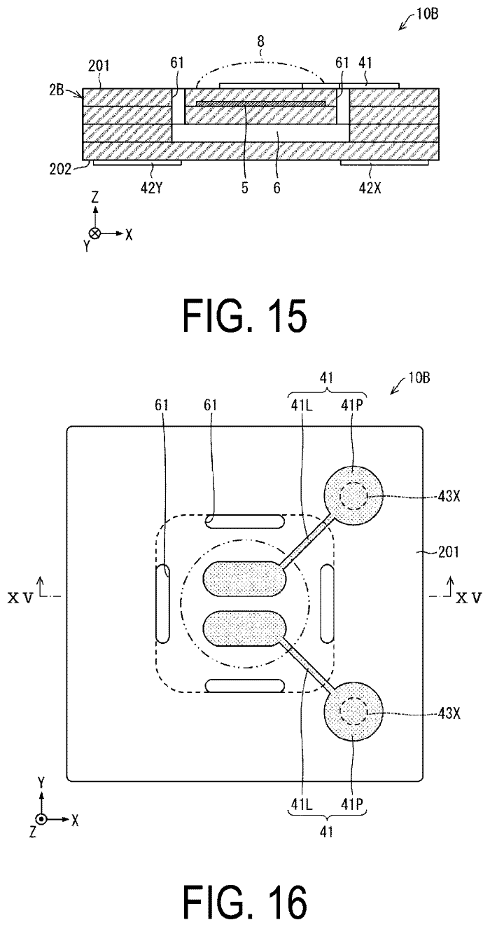

In the third embodiment, a substrate 10B according to another embodiment will be described with reference to FIGS. 15 and 16.

FIG. 15 is a cross-sectional view of the substrate 10B according to the third embodiment and is a cross-sectional view taken along the arrow line XV-XV in FIG. 16. FIG. 16 is a top view of the substrate 10B.

The substrate 10B is different from the substrate 10A of the second embodiment in that the substrate includes a through hole 61 extending from the cavity 6 to the first surface 201. The arrangement of the electrode wiring 41 on the first surface 201 is different from that in the substrate 10A. As illustrated in FIG. 16, the two first portions 41P located on the first surface 201 may be disposed along any one side of the substrate 10A.

The substrate 10B is different from the substrate 10A of the second embodiment in the shape of the second portion 41L of the electrode wiring 41. As illustrated in FIG. 16, the portion of the second portion 41L located in the gas sensitive body region 3 may have a pad shape.

The substrate 10B includes a base 2B. The base 2B includes four through holes 61. As illustrated in FIG. 16, the through holes 61 are located not to overlap the gas sensitive body region 3 and to overlap the cavity 6 in a perspective plan view. The number of the through holes 61 is not limited to four. The number, position, size, shape, and the like of the through holes 61 are not particularly limited and may be changed as appropriate as long as the through holes 61 do not overlap the gas sensitive body region 3 and penetrate from the cavity 6 to the surface of the substrate 10B.

As in the substrate 10B, since the through holes 61 are provided, the heat insulation property is improved as compared with the substrate 10A of the second embodiment, and the heat conduction from the heater 5 to the second surface 202 can be further reduced. The gas in the cavity 6 heated by the heater 5 is easily ventilated to the outside, and thus the temperature rise of the second surface 202 can be reduced. Since the through holes 61 are provided, the through holes 61 act as gas vent holes in the firing step of the substrate 10 including the insulation layer 20 made of a ceramic material, and thus the possibility of deformation in the manufacturing step can be reduced.

### Fourth Embodiment

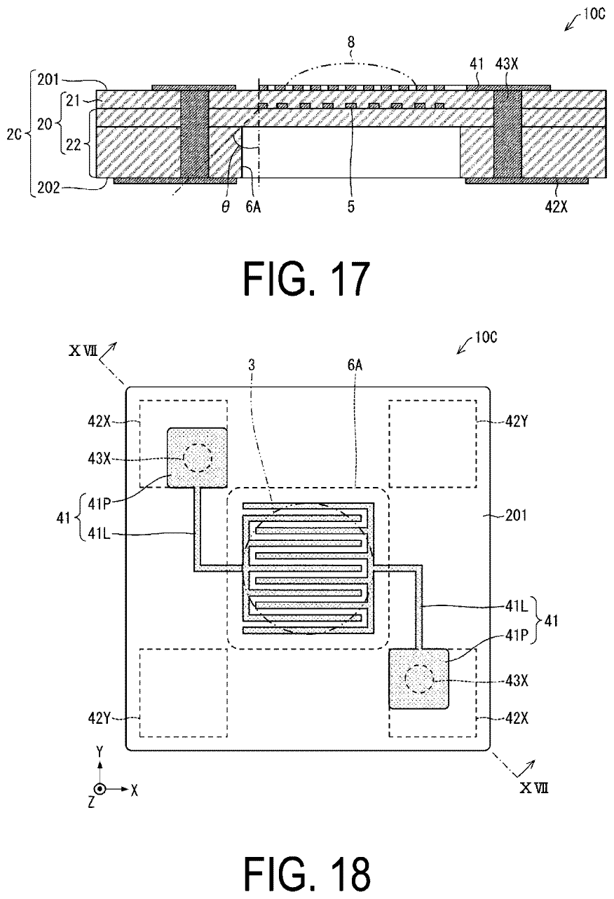

In the fourth embodiment, a substrate 10C according to another embodiment will be described with reference to FIGS. 17 and 18.

FIG. 17 is a cross-sectional view of the substrate 10C according to the fourth embodiment, and is a cross-sectional view taken along the arrow line XVII-XVII in FIG. 18. FIG. 18 is a top view of the substrate 10C. In FIG. 18, the shape of each member in a perspective plan view is indicated by dotted lines as appropriate for easy understanding of the positional relationship.

The substrate 10C is different from the substrate 10A of the second embodiment in that the cavity is a recessed portion 6A opening to the second surface 202. The substrate 10C includes the recessed portion 6A opening to the second surface 202 at a position overlapping the heater 5 in a perspective plan view. To be more specific, the second insulation layer of the substrate 10C includes the recessed portion 6A opening to the second surface 202. As illustrated in FIG. 17, the second insulation layer 22 is an insulation layer group including a plurality of insulation layers 20, and the insulation layers 20 other than the insulation layers where the heater 5 is located among the plurality of insulation layers 20 may have a through hole forming the recessed portion 6A. The shape of the recessed portion 6A may be rectangular in a plan view as illustrated in FIG. 18. The shape of the recessed portion 6A is not limited to a rectangle, and may be designed as appropriate.

When the cavity has a recessed shape as in the substrate 10C, the cavity can be made larger than that in the hollow structure as in the substrate 10A of second embodiment. Since the cavity is the open recessed portion 6A, heat is dissipated from the inner surface of the recessed portion 6A to the outside. This further improves the heat insulation property. The recessed shape is easier to manufacture than the hollow structure.

In the cross section illustrated in FIG. 17, the outer edge of the bottom surface of the recessed portion 6A may be located outside the outer edge of the heater 5. This configuration can improve the heat insulation property. In the cross section illustrated in FIG. 17, an angle θ formed by a line extending from the outer edge of the heater 5 in the Z axis direction and a line connecting the outer edge and the outer edge of the bottom surface of the recessed portion 6A may be 45 degrees or more. Since the recessed portion 6A is present in the range where most of heat from the heater 5 is transmitted, the heat insulation property can be improved.

Although FIGS. 17 and 18 illustrate an example in which the base 2C includes an recessed portion which is one large rectangle in a perspective plan view, the number, shape, and size of the recessed portions are not limited to this example. The shape of the recessed portion in a plan view may be circular, or may include a plurality of recessed portions as illustrated in FIGS. 11, 12, and 13. By providing a plurality of recessed portions, the strength of the substrate 10C is improved as compared with the case where one large recessed portion 1 is provided. A through hole extending from the bottom surface of the recessed portion 6A to the first surface 201 may be provided. Providing the through hole further improves the heat insulation property, and can further reduce the heat conduction from the heater 5 to the second surface 202. The gas of the recessed portion 6A heated by the heater 5 is easily ventilated to the outside, and the temperature rise of the second surface 202 can be reduced. Since the through hole is provided, the through hole acts as a gas vent hole in the baking step of the substrate 10C including the insulation layer 20 made of a ceramic material, and thus the possibility of deformation in the manufacturing step can be reduced.

### Fifth Embodiment

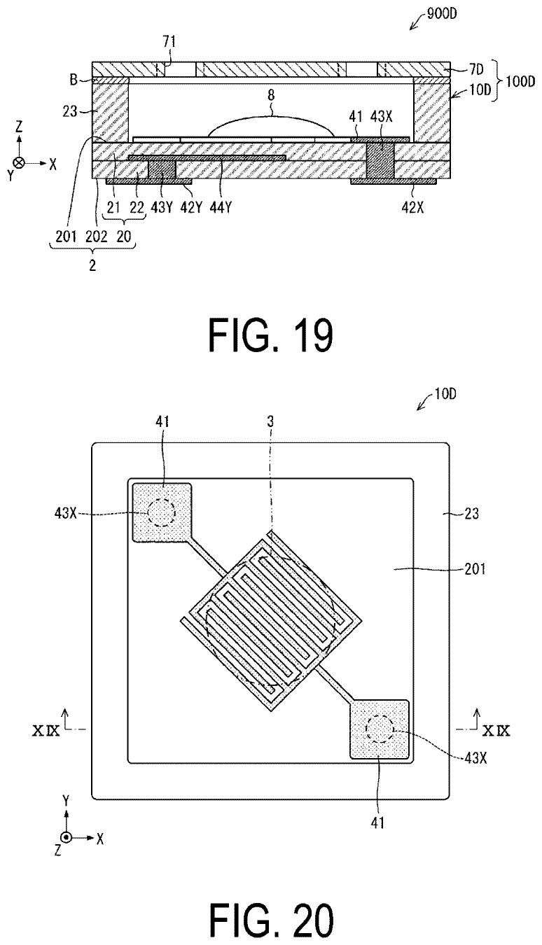

In the fifth embodiment, a gas sensor 900D, a package 100D, and a substrate 10D according to another embodiment of the first embodiment will be described with reference to FIGS. 19 to 21. The gas sensor 900D according to the fifth embodiment includes the package 100D and the gas sensitive body 8. The package 100D includes the substrate 10D and the lid 7D. The substrate 10D includes the base 2, an insulation frame body 23, the gas sensitive body region 3, the wiring 4, and the heater 5.

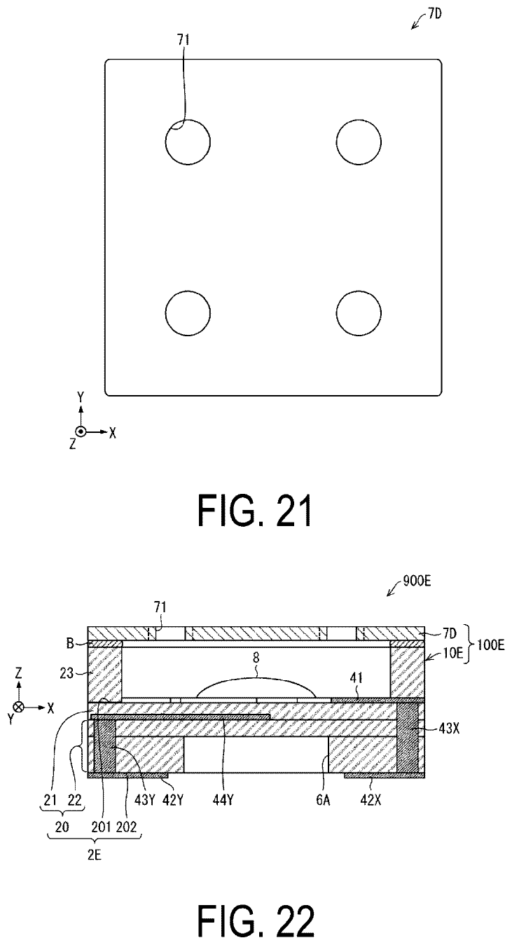

FIG. 19 is a sectional view of the gas sensor 900D, illustrating a state in which the bonding material B and the lid 7D are mounted to the cross-sectional view taken along the arrow line XVIX-XVII in FIG. 20. FIG. 20 is a top view of the substrate 10D. In FIG. 20, the shapes of the through conductors 43X in a perspective plan view are indicated by dotted lines for easy understanding of the positional relationship. FIG. 21 is a top view of the lid 7D.

The fifth embodiment is different from the first embodiment in that the gas sensor 900D includes the flat plate-shaped lid 7D and the substrate 10D includes the insulation frame body 23. The other points not particularly described below have the same configuration as that of the first embodiment.

In the fifth embodiment, the substrate 10D includes the insulation frame body 23 located on the first surface 201 of the base 2 to surround the gas sensitive body region 3. The length of the insulation frame body 23 in the Z axis direction is larger than the height of the gas sensitive body 8. The insulation frame body 23 may be an insulation layer made of the same ceramic material as that of the base 2. Since the substrate 10D includes the insulation frame body 23, a space capable of accommodating the gas sensitive body 8 can be formed, and the shape of the lid can be simplified. In the course of manufacturing the gas sensor 900D, the gas sensitive body 8 can be protected in a state where the gas sensitive body 8 is applied to the substrate 10D before the lid 7D is fixed.

The gas sensor 900D may include a flat plate-shaped lid 7D that covers the housing recessed portion formed by the base 2 and the insulation frame body 23. By forming the lid 7D in a flat plate shape, the manufacture is facilitated. As illustrated in FIG. 21, the lid 7D may include a plurality of through holes 71. With the plurality of through holes 71, breathability can be improved. The through holes 71 may be provided at positions not overlapping the gas sensitive body 8 in a perspective plan view. Not overlapping the gas sensitive body 8 in a perspective plan view can reduce the possibility that dust or the like that has entered from the through holes 71 adheres to the gas sensitive body 8.

The lid 7D may be made of a ceramic material. Since the lid 7D is made of ceramics, a difference in heat expansion between the lid 7D and the substrate 10D does not occur, and the possibility that the gas sensor 900D is deformed by heat stress due to the difference in heat expansion can be reduced. Therefore, the gas sensor 900D has high connection reliability between the gas sensitive body 8 and the electrode wiring 41 and between the external electrode 42X and the mounting substrate.

The lid 7D made of a ceramic material and the insulation frame body 23 may be bonded to each other using the bonding material B such as glass frits or resins. Alternatively, a frame-shaped metal film may be provided on the upper surface of the insulation frame body 23, and the insulation frame body 23 may be bonded to the frame-shaped metal film using a brazing material or solder. When the frame-shaped metal film and the lid 7D are bonded to each other with a brazing material, a metal film having the same configuration as the frame-shaped metal film may also be provided on the lower surface of the lid 7D. A nickel film may be formed on the surfaces of the frame-shaped metal film and the lid 7D by a plating method in order to improve bondability of the bonding using a brazing material.

### Sixth Embodiment

In the sixth embodiment, a gas sensor 900E, a package 100E, and a substrate 10E according to another embodiment will be described with reference to FIG. 22. The gas sensor 900E according to the sixth embodiment includes the package 100E and the gas sensitive body 8. The package 100E includes the substrate 10E and the lid 7D. The substrate 10E includes a base 2E, the insulation frame body 23, the gas sensitive body region 3, the wiring 4, and the heater 5.

FIG. 22 is a cross-sectional view of the gas sensor 900E, illustrating a cross section at the same position as in FIG. 19. In the sixth embodiment, the substrate 10E includes the recessed portion 6A opening to the second surface 202 at a position overlapping the heater 5 in a perspective plan view. To be more specific, the second insulation layer of the substrate 10C includes the recessed portion 6A opening to the second surface 202.

By providing the recessed portion 6A, heat conduction from the heater 5 to the second surface 202 can be reduced.

### Seventh Embodiment

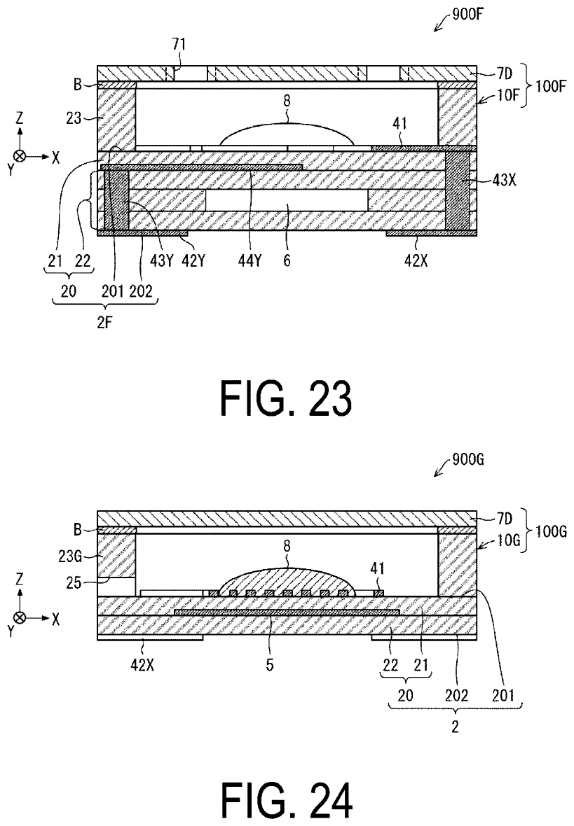

In the seventh embodiment, a gas sensor 900F, a package 100F, and a substrate 10F according to another embodiment of the sixth embodiment illustrated in FIG. 22 will be described with reference to FIG. 23. The gas sensor 900F according to the seventh embodiment includes the package 100F and the gas sensitive body 8. The package 100F includes the substrate 10F and the lid 7D. The substrate 10F includes a base 2F, the insulation frame body 23, the gas sensitive body region 3, the wiring 4, and the heater 5.

FIG. 23 is a cross-sectional view of the gas sensor 900F. In the seventh embodiment, the substrate 10F includes the hollow cavity 6 instead of the recessed portion 6A of the substrate 10E of the sixth embodiment. Even this configuration can reduce the conduction of heat from the heater 5 to the second surface 202.

### Eighth Embodiment

In the eighth embodiment, a gas sensor 900G, a package 100G, and a substrate 10G according to another embodiment will be described with reference to FIGS. 24 to 26. The gas sensor 900G according to the eighth embodiment includes the package 100G and the gas sensitive body 8. The package 100G includes the substrate 10G and the lid 7D. The substrate 10G includes the base 2, the insulation frame body 23G, the gas sensitive body region 3, the wiring 4, and the heater 5.

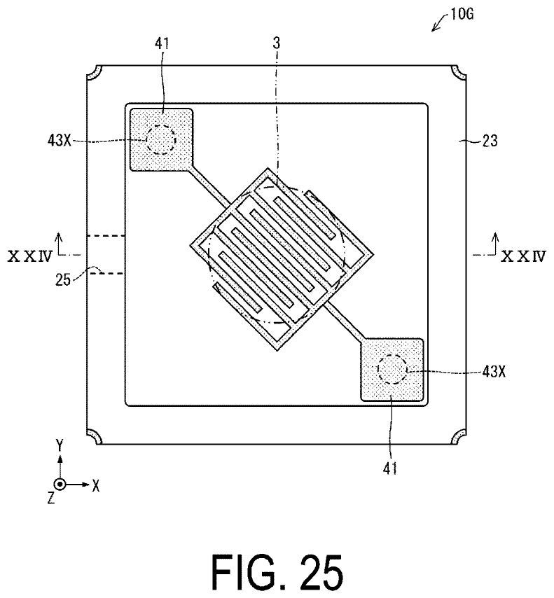

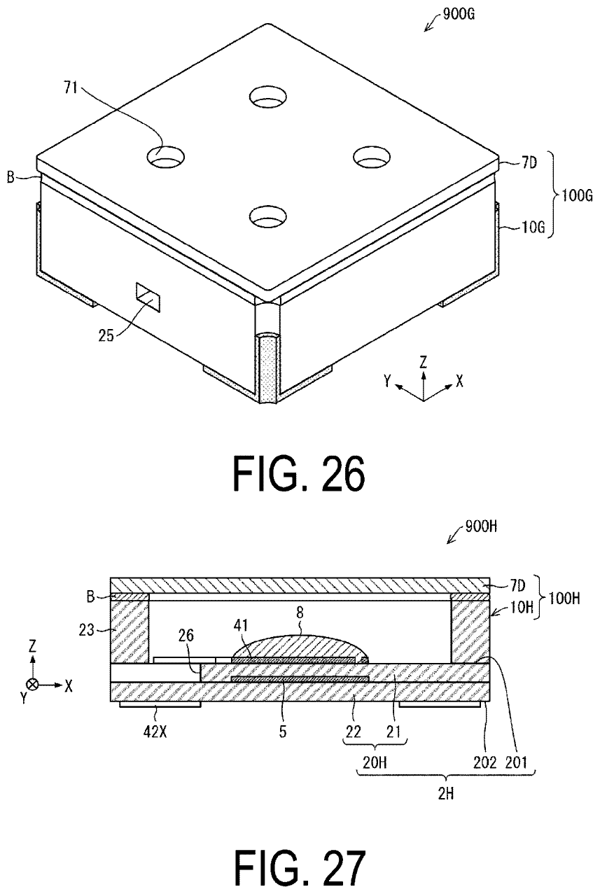

FIG. 24 is a cross-sectional view of the gas sensor 900G, and illustrates a cross-sectional view taken along the arrow line XXIV-XXIV in FIG. 25 in a state where the bonding material B and the lid 7D are mounted to the substrate 10G. FIG. 25 is a top view of the substrate 10G. FIG. 26 is a perspective view of the gas sensor 900G. In FIG. 25, the shapes of the through conductors 43X and the through hole 25 in a perspective plan view are indicated by dotted lines for easy understanding of the positional relationship.

In the eighth embodiment, the substrate 10G includes a through hole penetrating from the inner side surface of the insulation frame body 23G to the outer surface of the substrate 10G. To be more specific, the insulation frame body 23G of the substrate 10G includes the through hole 25 penetrating from the outer surface to the inner surface of the insulation frame body 23G. In the example illustrated in FIG. 25, one through hole 25 is located at the center of one side of the insulation frame body 23G, but the position, number, and shape of the through hole 25 are not limited thereto.

The gas heated by the heater 5 disposed below the gas sensitive body 8 is emitted to the outside from the through hole 71 of the lid 7D by the ascending air current. Then, the internal pressure of the package 100G is lowered, and the external gas flows into the package 100G from the through hole 25 of the insulation frame body 23G. That is, a series of gas flows is formed. This can enhance the gas breathability of the package 100G, and can enhance the gas detection accuracy of the gas sensitive body 8.

### Ninth Embodiment

In the ninth embodiment, a gas sensor 900H, a package 100H, and a substrate 10H according to another embodiment will be described with reference to FIGS. 27 to 29. The gas sensor 900H according to the ninth embodiment includes the package 100H and the gas sensitive body 8. The package 100H includes the substrate 10H and the lid 7D. The substrate 10H includes a base 2H, the insulation frame body 23, the gas sensitive body region 3, the wiring 4, and the heater 5.

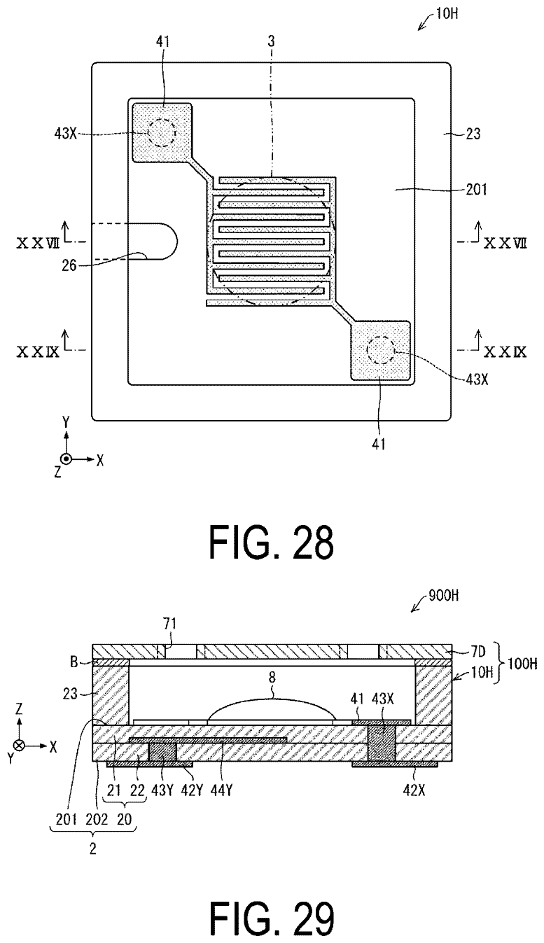

FIG. 27 is a cross-sectional view of the gas sensor 900H, and illustrates a cross-sectional view taken along the arrow line XXVII-XXVII in a state where the bonding material B and the lid 7D are mounted to the substrate 10H of FIG. 28. FIG. 28 is a top view of the substrate 10H. FIG. 29 is a cross-sectional view of the gas sensor 900H, and is a cross-sectional view taken along the arrow line XXIX-XXVIIX in a state where the bonding material B and the lid 7D are mounted to the substrate 10H of FIG. 28. In FIG. 28, the shapes of the through conductors 43X and the through hole in a perspective plan view are partially indicated by dotted lines for easy understanding of the positional relationship.

In the ninth embodiment, the substrate 10H includes a through hole penetrating from the first surface 201 to the outer surface of the substrate 10H. In other words, the through hole of the substrate 10H penetrates from the outer surface of the substrate 10H to the housing space of the gas sensitive body 8 in the package 100H. The through hole is open to the side surface of the substrate 10H and the first surface 201. For example, the first insulation layer 21 of the substrate 10H may include a recessed portion 26, and the recessed portion may form the through hole in the substrate 10H.

In the example illustrated in FIGS. 27 and 28, one through hole is located at the center of one side of the substrate 10H, but the position, number, and shape of the through hole are not limited thereto.

Even in the configuration of the gas sensor 900H according to the ninth embodiment, the gas breathability of the package 100G can be increased, and the gas detection accuracy of the gas sensitive body 8 can be enhanced as the gas sensor 900G according to the eighth embodiment.

### Tenth Embodiment

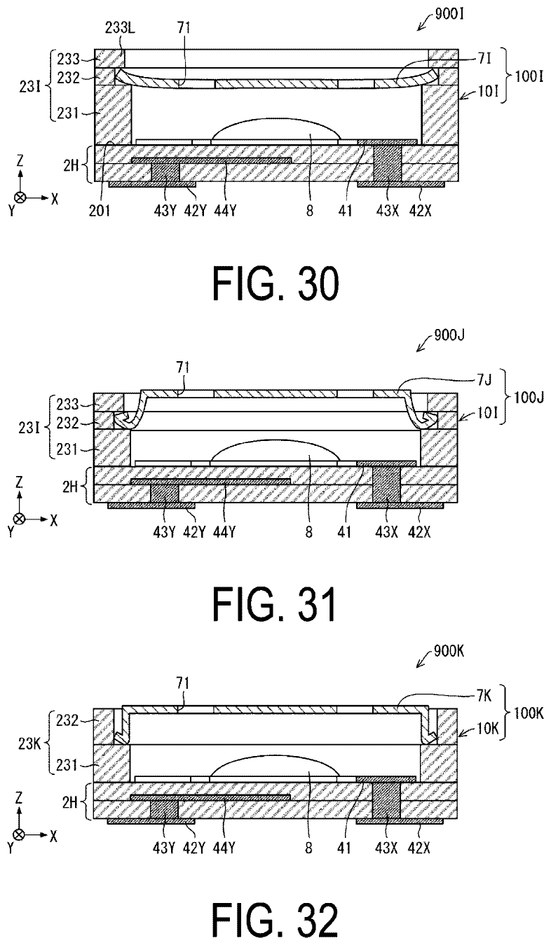

In the tenth embodiment, a gas sensor 900I, a package 100I, and a substrate 10I according to another embodiment will be described first with reference to FIG. 30. FIG. 30 is a cross-sectional view of the gas sensor 900I according to another embodiment. The gas sensor 900I according to the tenth embodiment includes the package 100I and the gas sensitive body 8. The package 100I includes the substrate 10I and a lid 7I. The substrate 10I includes a base 2H, an insulation frame body 23I, the gas sensitive body region 3, the wiring 4, and the heater 5.

The insulation frame body 23I includes a first frame body 231 located on the first surface 201 of the base 2H to surround the gas sensitive body region 3, a second frame body 232 located on the upper surface of the first frame body 231, and a third frame body 233 located on the upper surface of the second frame body 232. In a perspective plan view, the inner edge of the second frame body 232 is located outside the inner edge of the third frame body 233. In a perspective plan view, the inner edge of the third frame body may overlap the inner edge of the first frame body 231 or may be located outside the inner edge of the first frame body 231. In the third frame body 233, a portion protruding inward of the substrate 10I from the second frame body 232 functions as a limiting portion that limits the movement of the lid 7I in a direction away from the substrate 10I. In other words, the insulation frame body 231 of the substrate 10I includes a limiting portion 233L that limits the movement of the lid 7I in the direction away from the substrate.

The lid 7I is an elastic metal lid and may include the through hole 71. In the package 100I, the edge portion of the lid 7I is located on the inner side of the insulation frame body 23I. The edge portion of the lid 7I is located below the limiting portion 233L.

With this configuration, the lid 7I is held on the substrate 10I by the elasticity of the lid 7I and the limiting portion 233L that limits the movement of the lid 7I. This allows the lid 7I to be held by the substrate 10I without being bonded to the substrate 10I using a bonding material such as resins.

Next, a lid 7J according to according to another embodiment will be described with reference to FIG. 31. FIG. 31 is a cross-sectional view of a gas sensor 900J according to another embodiment. The gas sensor 900J illustrated in FIG. 31 has the same configuration as the gas sensor 900I illustrated in FIG. 30 except for the shape of the lid 7J. The lid 7J is also an elastic metal lid. The lid 7J includes a curved portion or a bent portion formed by bending the end portion thereof outward. The lid 7J including a curved portion or a bent portion facilitates insertion of the lid 7J into the insulation frame body 23I.

Next, an insulation frame body 23K and a lid 7K according to another embodiment will be described with reference to FIG. 32. FIG. 31 is a cross-sectional view of a gas sensor 900K according to another embodiment. The insulation frame body 23K illustrated in FIG. 32 includes the first frame body 231 located on the first surface 201 of the base 2H to surround the gas sensitive body region 3, and the second frame body 232 located on the upper surface of the first frame body 231. In a substrate 10K, the inner edge of the second frame body 232 located in the uppermost layer is located outside the inner edge of the third frame body 233. With this configuration, the lid 7K can be easily inserted into the insulation frame body 23K.

The lid 7K is an elastic metal lid. The lid 7K can be held on the substrate 10K by the elasticity of the lid 7K without using resins or the like. The lid 7K has a cap shape and includes a curved portion or a bent portion formed by bending the end portion thereof outward. The lid 7K including a curved portion or a bent portion facilitates insertion of the lid 7K into the insulation frame body 23K.

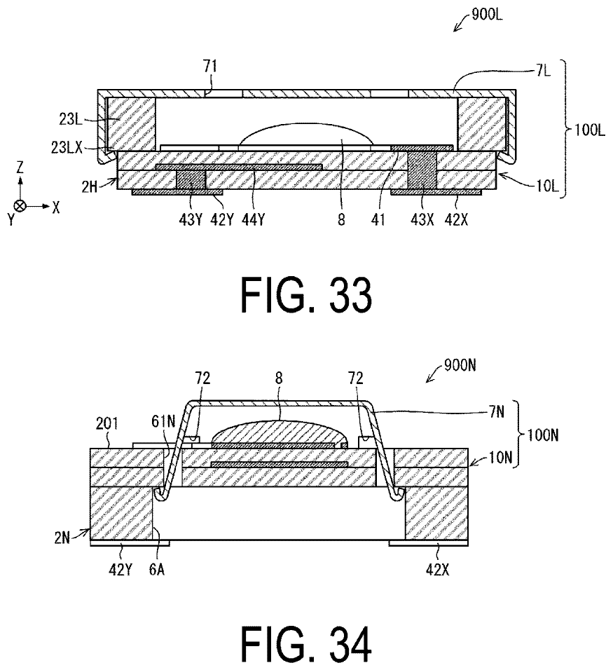

Next, an insulation frame body 23L and a lid 7L according to another embodiment will be described with reference to FIG. 33. FIG. 33 is a cross-sectional view of a gas sensor 900L according to another embodiment. The insulation frame body 23L illustrated in FIG. 33 is located on the first surface 201 of the base 2H to surround the gas sensitive body region 3. In a perspective plan view, the outer edge of the insulation frame body 23L is located outside the outer edge of the base 2H.

The lid 7L is an elastic metal lid. The lid 7L has a cap shape capable of housing the insulation frame body 23L and includes a bent portion formed by bending the end portion thereof inward.

The bent portion of the lid 7L can be engaged with a protruding portion 23LX of the insulation frame body 23L, the protruding portion 23LX protruding outward from the base 2H. That is, in the insulation frame body 23L, the protruding portion 23LX protruding outward from the base 2H functions as a limiting portion that limits the movement of the lid 7I in a direction away from the substrate 10I.

With this configuration, the lid 7L can be held on a substrate 10L without using a bonding material such as resins. Long-term reliability is improved by not using a bonding material that may be deteriorated by an external environment such as gas or humidity.

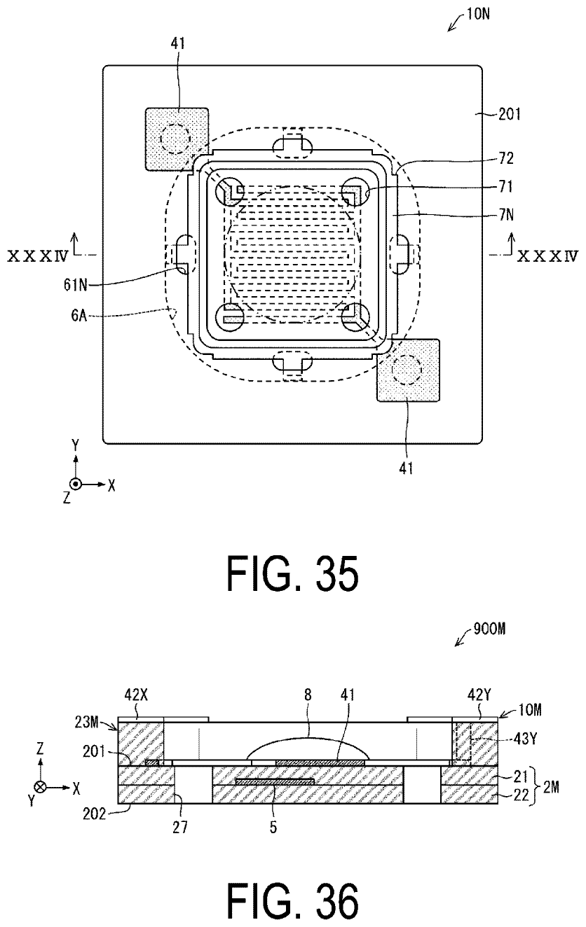

Next, a substrate 10N and a lid 7N according to another embodiment will be described with reference to FIGS. 34 and 35. FIG. 34 is a cross-sectional view of a gas sensor 900N according to another embodiment. FIG. 34 is a cross-sectional view taken along the arrow line XXXIV-XXXIV in FIG. 35. FIG. 35 is a top view of the gas sensor 900N. A base 2N of the substrate 10N illustrated in FIGS. 34 and 35 includes the recessed portion 6A. The base 2N includes a through hole 61N penetrating from the bottom surface of the recessed portion 6A to the first surface 201 of the base 2N.

The lid 7N is an elastic metal lid. The lid 7N includes a cap-shaped body portion covering the gas sensitive body 8 and a claw extending from the body portion. The claw is inserted into the through hole 61N and is locked to the bottom surface of the recessed portion 6A. The lid 7N may include the through hole 71. The lid 7N may include a cutout 72 in the body portion, for example, in order to prevent short with the electrode wiring 41 and to improve breathability.

With this configuration, the lid 7N can be held on the substrate 10N without using a bonding material such as resins. Long-term reliability is improved by not using a bonding material that may be deteriorated by an external environment such as gas or humidity.

In the examples illustrated in the first to tenth embodiments described above, each of the gas sensors 900, 900D, 900E, 900F, 900G, 900H, 900I, 900J, 900K, 900L, and 900N includes a lid. However, the lid is not an essential component of the gas sensor. That is, it should be understood that an aspect in which the gas sensor does not include a lid is also within the scope of the present disclosure.

### Eleventh Embodiment

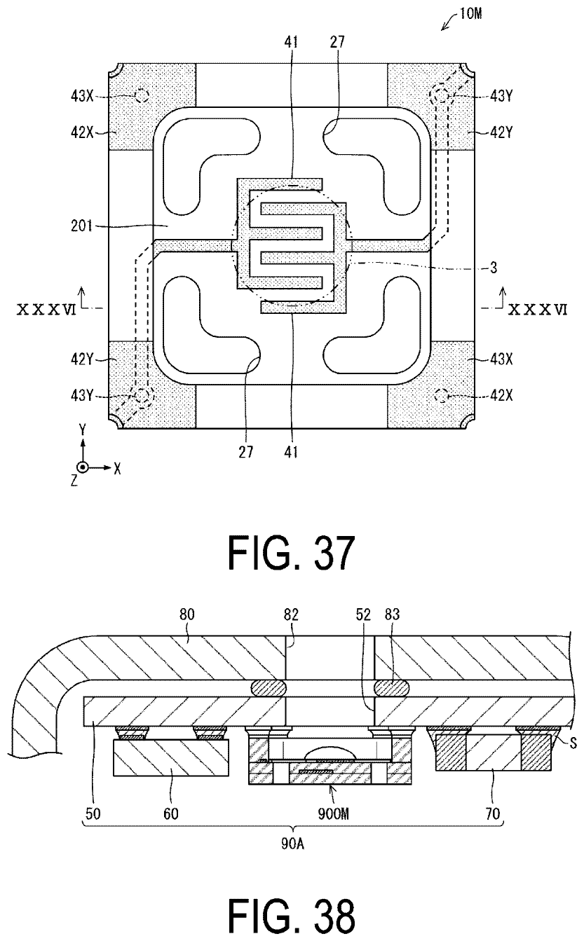

In the eleventh embodiment, a gas sensor 900M and the substrate 10M according to another embodiment of the fifth embodiment will be described with reference to FIGS. 36 and 37. FIG. 36 is a cross-sectional view of the gas sensor 900M, and illustrates a cross-sectional view taken along the arrow line XXXVI-XXXVI in FIG. 37. FIG. 37 is a top view of the substrate 10M. The gas sensor 900M according to the eleventh embodiment includes the substrate 10M and the gas sensitive body 8. The substrate 10M includes a base 2M, the insulation frame body 23M, the gas sensitive body region 3, the wiring 4, and the heater 5.

The substrate 10M includes the first external electrode 42X and the second external electrode 42Y on the insulation frame body 23M. That is, the gas sensitive body region 3 is different from the other embodiments described above in that the gas sensitive body region 3 is located between the surface on which the first external electrode 42X and the second external electrode 42Y are located and the surface on which the heater 5 is located. The first external electrode 42X is electrically connected to the electrode wiring 41 via the through conductor 43X. The second external electrode 42Y is electrically connected to the heater 5 via the through conductor 43Y.

Since the first external electrode 42X and the second external electrode 42Y are located on the insulation frame body 23M, surface mounting is possible on the upper surface side of the insulation frame body 23M. Since the first external electrode 42X and the second external electrode 42Y are located on the insulation frame body 23M, the distances from the heater 5 to the two external electrodes are increased. This reduces the heat effect on the electrically conductive bonding material when the gas sensor 900M is mounted. In this case, the substrate 10M can also serve as a lid, and another lid is not necessary to be provided. This provides effects such as simplification of the manufacturing step, reduction in manufacturing cost, and reduction in height of the gas sensor.

The base 2M of the substrate 10M includes the through hole 27 for ventilation. To be specific, the base 2M includes a through hole penetrating from the first surface 201 to the outer surface of the substrate 10M. To be more specific, the base 2M portion includes the through hole 27 penetrating from the first surface 201 to the second surface 202. The base 2M may include a plurality of through holes 27. With the plurality of through holes 27, breathability can be improved.

The through hole 27 may be located at a corner portion of a rectangular region inside the insulation frame body 23M in a plan view. By disposing the through hole 27 at the corner portion having a large area, a decrease in the strength of the base 2M can be reduced. The through hole 27 may have an L-shape extending from each corner portion along the inner surface of the insulation frame body 23M. Since the through hole 27 has an L-shape, the area of the through hole 27 can be increased while securing the area of the region where the gas sensitive body region 3 and the electrode wiring 41 are disposed.

As the size of the through hole 27 increases, the heat conduction from the heater 5 to the external electrode 42 decreases, and the breathability of the gas to be detected increases, and thus the sensing accuracy of the gas sensor increases. By increasing the size of the through hole 27, the portion where the heater 5 and the outer peripheral portion of the substrate 10M are connected to each other is reduced. This makes it easier for the heat of the heater 5 to be transferred to the gas sensitive body 8, thereby improving the heating efficiency of the gas sensitive body 8.

Even in the substrate 10M of the eleventh embodiment, the distances between the heater 5 and the first surface 201 may be smaller than the distance between the heater 5 and the second surface 202. Since the first insulation layer 21 is thinner than the second insulation layer 22, the heat from the heater 5 is more easily transferred to the first surface 201 side where the gas sensitive body 8 is located than to the second surface 202 side connected to the outside.

The distance between the heater 5 and the first surface 201 may be 10 μm or more and 100 μm or less. This configuration can reduce the possibility that the heater 5 is exposed to the first surface 201. The distance between the heater 5 and the second surface 202 may be 100 μm or more. This configuration can reduce the temperature rise of the second surface 202.

A through hole may be provided, the through hole penetrating from the inner side surface of the insulation frame body 23M to the outer surface of the substrate 10M. The outer surface of the substrate 10M includes the outer side surface of the substrate 10M and the second surface 202. That is, the substrate 10M may further include a through hole penetrating from the inner side surface to the outer side surface of the insulation frame body 23M. This configuration can increase the gas breathability of the package 100G and can increase the gas detection accuracy of the gas sensitive body 8.

Alternatively, the substrate 10M may further include a through hole penetrating from the first surface 201 to the outer side surface of the substrate 10M (the side surface of the base 2M) as illustrated in FIG. 27. This configuration can increase the gas breathability of the package 100G and can increase the gas detection accuracy of the gas sensitive body 8.

FIG. 38 is a cross-sectional view of a gas sensor module 90A including the gas sensor 900M. FIG. 38 illustrates a state in which the gas sensor module 90A is mounted to a housing 80 of an electronic device or the like.

The gas sensor module 90A includes the mounting substrate 50 and the gas sensor 900M. In the example illustrated in FIG. 38, the gas sensor 900M, the semiconductor IC 60, and the capacitor 70 are mounted on the mounting substrate 50. The gas sensor 900, the semiconductor IC 60, and the capacitor 70 are surface-mounted on the mounting substrate 50 by solder S, respectively. The mounting substrate 50 includes a through hole 52 at a position corresponding to the gas sensitive body 8. The housing 80 may also include a through hole 82 at a position corresponding to the gas sensitive body 8 or the through hole 52. As illustrated in FIG. 38, the gas sensor 900M can be directly soldered on the mounting substrate 50 together with electronic components such as the semiconductor IC 60 and the capacitor 70 to form the gas sensor module 90A.

In FIG. 38, the gas sensor 900M and the like are mounted on the surface of the mounting substrate 50 on the side opposite to the housing 80. When the through hole 82 of the housing 80 and the through hole 52 of the mounting substrate 50 are large and have sufficient breathability, the substrate of the gas sensor 900M may not include a through hole.

Alternatively, the gas sensor 900M and the electronic components may be mounted on the surface of the mounting substrate 50 on the housing 80 side. In this case, the mounting substrate 50 may not include the through hole 52.

### Twelfth Embodiment

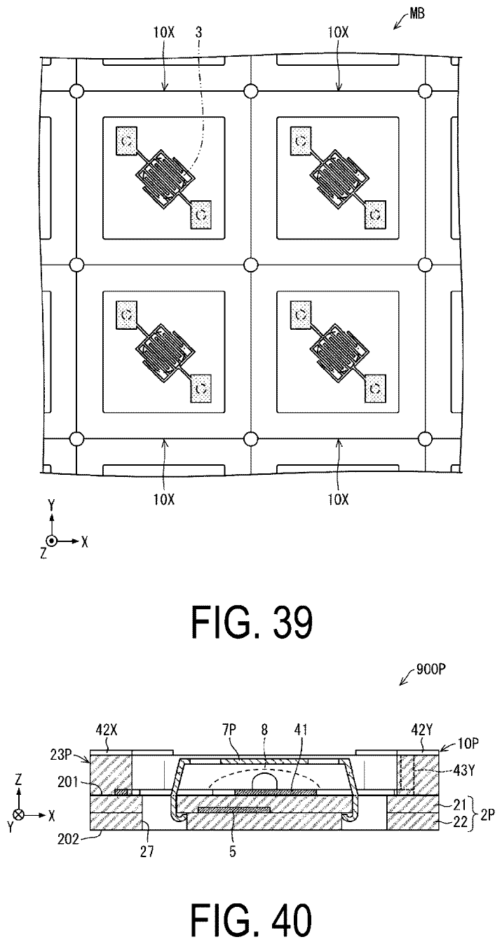

In the twelfth embodiment, a method for manufacturing the gas sensor 900 described in the first embodiment will be described with reference to FIG. 39. FIG. 39 is a schematic plan view illustrating a part of a mother board MB according to an exemplary embodiment.

First, a mother board including a plurality of substrate regions 10X corresponding to the substrate 10 is prepared. As shown in FIG. 39, the mother board MB has a plurality of substrate regions 10X corresponding to the substrates 10 of the first embodiment.

When the base 2 of the substrate 10 is made of an aluminum oxide sintered body, the mother board MB is produced as follows. An appropriate organic binder, a solvent, and the like are added to and mixed with a raw material powder of aluminum oxide, silicon oxide or the like to produce a slurry. A ceramic green sheet for an insulation layer 20 of the substrate 2 is produced by forming the slurry into a sheet shape by a doctor blade method, a calendar roll method, or the like. The ceramic green sheet for the insulation layer 20 is subjected to appropriate punching processing for forming a through hole for the through conductor 43 or the like, and a metal paste to be the through conductor 43 is filled therein. A metal paste to be the electrode wiring 41, the external electrode 42, the interlayer conductor 44, a frame-shaped metal film M, and the heater 5 is applied by the method such as a screen printing method. Subsequently, a plurality of the ceramic green sheets for the insulation layer are layered to produce a laminate body. The laminate body is, then, fired at a high temperature (from about 1300° C. to 1600° C.), whereby the mother board MB is produced.

After the mother board MB is prepared, the gas sensitive body 8 is formed. The gas sensitive body 8 is obtained by forming a semiconductor material to be the gas sensitive body 8 in a thin film or a thick film in the gas sensitive body region 3 in each substrate region 10X of the mother board MB, and then sintering the semiconductor material at a high temperature of about 500° C. to 800° C.

After the gas sensitive body 8 is formed, the lid 7 is fixed to each substrate region 10X by, for example, resin-bonding.

Then, the mother board MB is singulated by a method such as snap breaking or dicing, whereby a plurality of gas sensors 900 are obtained.

The above-described method enables mass production of the substrate 10 with high efficiency. Since the gas sensor 900 includes a simple structure in which the gas sensitive body 8 is directly disposed on the substrate 10, the gas sensor 900 can be manufactured with a simple process.

### Thirteenth Embodiment

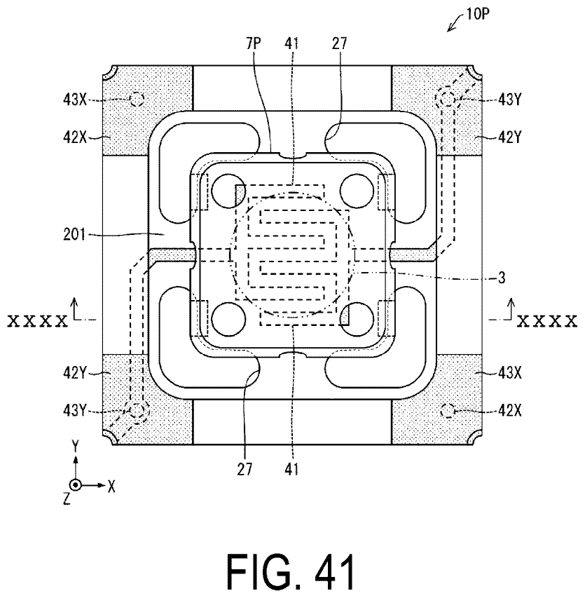

In the thirteenth embodiment, an aspect of a package and a gas sensor each including a substrate in which the first external electrode 42X and the second external electrode 42Y are located on the insulation frame body 23P, and a lid will be described. A gas sensor 900P as an example of such an aspect will be described with reference to FIGS. 40 and 41. FIG. 40 is a cross-sectional view of the gas sensor 900P, and illustrates a cross-sectional view taken along the arrow line XXXX-XXXX in FIG. 41. FIG. 41 is a top view of the gas sensor 900P.

A substrate 10P has the same configuration as the substrate 10M illustrated in FIG. 36 except that the substrate 10P includes a step capable of locking a lid 7P. The step for locking the lid 7P may be formed on the inner side surface of the through hole 27 as illustrated in FIG. 40.

The lid 7P may be made of metal. The lid 7P includes a cap-shaped body portion covering the gas sensitive body 8 and a claw extending from the body portion. The claw is inserted into the through hole 27 and is locked to the bottom surface of the first insulation layer. The lid 7P may include a through hole on an upper surface thereof.

The example illustrated in FIGS. 40 and 41 is merely an example, and the configuration is not limited to the above-described example. For example, in the example illustrated in FIG. 30, 31, or 32, the package and the gas sensor may be configured such that the first external electrode 42X and the second external electrode 42Y are located on the insulation frame body. Alternatively, the package and the gas sensor may include the substrate 10M illustrated in FIGS. 36 and 37 and a flat plate-shaped lid that is joined to the insulation frame body 23M other than the first external electrode 42X and the second external electrode 42Y located at the corner portions. The lid may be made of ceramic.

Even when the first external electrode 42X and the second external electrode 42Y are located on the insulation frame body 23P, the provision of the lid can protect the gas sensitive body 8.

### CONCLUSION

(1) A substrate according to a first aspect of the present disclosure includes a base including a plurality of insulation layers made of a ceramic material and including a first surface and a second surface located on a side opposite to the first surface, a gas sensitive body region located on the first surface and in which a gas sensitive body is disposed, an electrode wiring located in the gas sensitive body region and connected to the gas sensitive body, a heater located between the insulation layers and located overlapping the gas sensitive body region in a perspective plan view, and an external electrode located on the second surface and electrically connected to the electrode wiring or the heater.
(2) A substrate according to a second aspect of the present disclosure is the substrate according to the first aspect, in which a distance between the heater and the first surface is smaller than a distance between the heater and the second surface.
(3) A substrate according to a third aspect of the present disclosure is the substrate according to the first or second aspect, in which a distance between the heater and the first surface is 10 μm or more and 100 μm or less.
(4) A substrate according to a fourth aspect of the present disclosure is the substrate according to any one of the first to third aspects, in which a distance between the heater and the second surface is 100 μm or more.
(5) A substrate according to a fifth aspect of the present disclosure is the substrate according to any one of the first to fourth aspects, in which the base includes a cavity between the heater and the second surface.
(6) A substrate according to a sixth aspect of the present disclosure is the substrate according to the fifth aspect, in which the cavity is a recessed portion opening in the second surface.
(7) A substrate according to a seventh aspect of the present disclosure is the substrate according to the fifth or sixth aspect, in which the base further includes a through hole extending from the cavity to the first surface.
(8) A substrate according to an eighth aspect of the present disclosure is the substrate according to any one of the first to seventh aspects, further including, on the first surface, an insulation frame body located surrounding the gas sensitive body region.
(9) A substrate according to a ninth aspect of the present disclosure is the substrate according to the eighth aspect, in which the base includes a recessed portion located overlapping the heater in a perspective plan view and opening in the second surface.
(10) A substrate according to a tenth aspect of the present disclosure is the substrate according to the eighth aspect, further including a through hole penetrating from an inner side surface of the insulation frame body to an outer surface of the substrate.
(11) A substrate according to an eleventh aspect of the present disclosure is the substrate according to any one of the first to tenth aspects, further including a through hole penetrating from the first surface to an outer surface of the substrate.
(12) A substrate according to a twelfth aspect of the present disclosure includes a base including a plurality of insulation layers made of a ceramic material and including a first surface, a gas sensitive body region located on the first surface and in which a gas sensitive body is disposed, an insulation frame body located surrounding the gas sensitive body region on the first surface, an electrode wiring located in the gas sensitive body region and connected to the gas sensitive body, a heater located between the insulation layers and located overlapping the gas sensitive body region in a perspective plan view, and an external electrode located on the insulation frame body and electrically connected to the electrode wiring or the heater.
(13) A substrate according to a thirteenth aspect of the present disclosure is the substrate according to the twelfth aspect, further including a through hole penetrating from the first surface to an outer surface of the base.
(14) A substrate according to a fourteenth aspect of the present disclosure is the substrate according to the twelfth or thirteenth aspect, in which the base includes a second surface located on a side opposite to the first surface, and a distance between the heater and the first surface is smaller than a distance between the heater and the second surface.
(15) A substrate according to a fifteenth aspect of the present disclosure is the substrate according to the fourteenth aspect, in which a distance between the heater and the first surface is 10 μm or more and 100 μm or less.
(16) A substrate according to a sixteenth aspect of the present disclosure is the substrate of the fourteenth or fifteenth aspect, in which a distance between the heater and the second surface is 100 μm or more.
(17) A substrate according to a seventeenth aspect of the present disclosure is the substrate according to any one of the twelfth to sixteenth aspects, further including a through hole penetrating from an inner side surface of the insulation frame body to an outer surface of the substrate.
(18) A substrate according to an eighteenth aspect of the present disclosure is the substrate according to any one of the twelfth to sixteenth aspects, further including a through hole penetrating from the first surface to an outer surface of the substrate.
(19) A package according to a nineteenth aspect of the present disclosure includes the substrate according to any one of the first to eleventh aspects and a lid.
(20) A package according to a twentieth aspect of the present disclosure is the package according to the nineteenth aspect, in which the lid is made of metal.
(21) A package according to a twenty first aspect of the present disclosure is the package according to the nineteenth aspect, in which the lid is made of a ceramic material.
(22) A package according to a twenty second aspect of the present disclosure includes the substrate according to the eighth aspect and a lid, in which the lid is an elastic metal lid, and an edge portion of the lid is located inside the insulation frame body.
(23) A package according to a twenty third aspect of the present disclosure is the package according to the twenty second aspect, in which the insulation frame body includes a limiting portion that limits a movement of the lid in a direction away from the substrate.
(24) A gas sensor according to a twenty fourth aspect of the present disclosure includes the package according to any one of the nineteenth to twenty third aspects, and a gas sensitive body.
(25) A gas sensor according to a twenty fifth aspect of the present disclosure includes the substrate according to any one of the twelfth to eighteenth aspects and a gas sensitive body.
(26) A gas sensor module according to a twenty sixth aspect of the present disclosure includes the gas sensor according to the twenty fourth aspect and a mounting substrate, in which the gas sensor is surface-mounted on the mounting substrate via a conductive bonding material.
(27) A gas sensor module according to a twenty seventh aspect of the present disclosure includes the gas sensor according to the twenty fifth aspect and a mounting substrate, in which the gas sensor is surface-mounted on the mounting substrate via a conductive bonding material.
(28) A gas sensor module according to a twenty eighth aspect of the present disclosure is the gas sensor module according to the twenty seventh aspect, in which the mounting substrate includes, at a position facing the gas sensitive body, a through hole that allows inflow of gas from an outside.
(29) A method for manufacturing a gas sensor according to a twenty ninth aspect of the present disclosure includes preparing a mother board including a plurality of substrate regions corresponding to the substrate according to any one of the first to eighteenth aspects, forming a gas sensitive body in the gas sensitive body region, and singulating the mother board.

The invention according to the present disclosure has been described above based on the drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, note that a person skilled in the art can easily make different variations or modifications based on the present disclosure. Note that these variations or modifications are within the scope of the present disclosure.

### REFERENCE SIGNS

10, 10A, 10B, 10C, 10D, 10E, 10F, 10G, 10H, 10I, 10K, 10L, 10M, 10N Substrate 2, 2A, 2B, 2C, 2E, 2H, 2M Base
20 Insulation layer
21 First insulation layer
22 Second insulation layer
23, 23G, 23I, 23K, 23L, 23M Insulation frame body
233L Limiting portion
25, 27 Through hole
3 Gas sensitive body region
4 Wiring
41 Electrode wiring
42 External electrode
43, 43X, 43X1, 43X2, 43Y Through conductor
44, 44X Interlayer conductor
44Y Heater terminal (interlayer conductor)
5 Heater
6 Cavity
61 Through hole
7, 7D, 7I, 7J, 7K, 7L, 7N, 7P Lid
71 Through hole
8 Gas sensitive body
B Bonding material
100, 100D, 100E, 100F, 100G, 100H, 100I Package
MB Mother board
10X Substrate region
900, 900D, 900E, 900F, 900G, 900H, 900I, 900J, 900K, 900L, 900M, 900N, 900P Gas sensor 90, 90A Gas sensor module

1. A substrate comprising:
a base comprising a plurality of insulation layers made of a ceramic material and comprising a first surface and a second surface located on a side opposite to the first surface;
a gas sensitive body region located on the first surface and in which a gas sensitive body is disposed;
an electrode wiring located in the gas sensitive body region and connected to the gas sensitive body;
a heater located between the insulation layers and located overlapping the gas sensitive body region in a perspective plan view; and
an external electrode electrically connected to the electrode wiring or the heater.

2. The substrate according to claim 1, wherein a distance between the heater and the first surface is smaller than a distance between the heater and the second surface.

3. The substrate according to claim 1, wherein
the base comprises a cavity between the heater and the second surface, and
the cavity is located overlapping the heater in a top perspective view.

4. The substrate according to claim 3, wherein the cavity is a recessed portion opening in the second surface.

5. The substrate according to claim 3, wherein the base further comprises a through hole extending from the cavity to the first surface.

6. The substrate according to claim 1, further comprising a through hole penetrating from the first surface to an outer surface of the substrate.

7. The substrate according to claim 1, further comprising, on the first surface, an insulation frame body located surrounding the gas sensitive body region.

8. The substrate according to claim 7, further comprising a through hole penetrating from an inner side surface of the insulation frame body to an outer surface of the substrate.

9. The substrate according to claim 1, wherein the external electrode is located on the second surface.

10. The substrate according to claim 7, wherein the external electrode is located on the insulation frame body.

11. A package comprising:
the substrate according to claim 1; and
a lid.

12. A package comprising:
the substrate according to claim 7; and
a lid, wherein
the lid is an elastic metal lid, and
an edge portion of the lid is located inside the insulation frame body.

13. The package according to claim 12, wherein the insulation frame body comprises a limiting portion that limits a movement of the lid in a direction away from the substrate.

14. A gas sensor comprising:
the package according to claim 11; and
a gas sensitive body.

15. A gas sensor comprising:
the substrate according to claim 1; and
a gas sensitive body.

16. A gas sensor module comprising:
the gas sensor according to claim 14; and
a mounting substrate, wherein
the gas sensor is surface-mounted on the mounting substrate via a conductive bonding material.

17. A gas sensor module comprising:
a gas sensor comprising the substrate according to claim 10 and a gas sensitive body; and
a mounting substrate, wherein
the gas sensor is surface-mounted on the mounting substrate via a conductive bonding material, and
the mounting substrate comprises, at a position facing the gas sensitive body, a through hole that allows inflow of gas from an outside.

18. A method for manufacturing a gas sensor comprising:
preparing a mother board comprising a plurality of substrate regions corresponding to the substrate according to claim 1;
forming a gas sensitive body in the gas sensitive body region; and
singulating the mother board.
